(19) United States
(12) Patent Application Publication
(10) Pub. No.: US 2003/0220647 A1
(43) Pub. Date: Nov. 27, 2003

(54) Low profile acetabular reamer

(76) Inventors: Kevin McCallum, South Bend, IN (US); Kevin Cook, Warsaw, IN (US)

Correspondence Address: JACQUE R. WILSON, ZIMMER, INC., 345 E. MAIN ST., WARSAW, IN, 46580, US

(21) Appl. No.: 10/153,053

(22) Filed: May 21, 2002

(51) Int. Cl. A61B 17/32

(52) U.S. Cl. 606/81

(57) ABSTRACT

An orthopaedic reaming apparatus, more specifically a low profile acetabular reamer useful in the performance of a minimally invasive or traditional hip arthroplasty. The acetabular reamer of the present invention comprises a convex non-hemispherical cutting surface having a plurality of cutting teeth disposed thereon.

### BACKGROUND OF THE INVENTION

The present invention relates generally to orthopaedic surgical instruments, and more specifically to acetabular reaming devices.

### DESCRIPTION OF THE RELATED ART

An endoprosthetic hip generally comprises a femoral component and an acetabular component. The femoral component includes a spherical head that covers or replaces the natural head of the femur. The acetabular component generally includes a hemispherical cup that is mounted in the acetabular cavity. The cup comprises a bone contacting surface and an articulating surface. The articulating surface is hemispherically shaped and adapted to function as the socket portion of a ball and socket joint, while the artificial femoral component acts as the ball portion of such a joint, thus mimicking the behavior of a natural hip joint. The bone contacting surface, however, of the acetabular component is fixedly attached to the acetabular cavity of the patient.

In order for a surgeon to place an acetabular component in a patient's acetabular cavity, the surgeon must first prepare the patient's acetabular cavity to accept the implant. Generally, this portion of a hip arthroplasty is accomplished by use of acetabular reamers known in the art to enlarge and reshape or otherwise prepare the acetabular cavity of the patient. Prior art acetabular reamers generally include a hemispherical head having a cutting surface with a plurality of cutting edges. The heads of such instruments are usually available in a variety of sizes. The surgeon must choose an appropriate size reamer for use during the surgery. During the preparation of the acetabular cavity of the patient, the surgeon must insert the entire hemispherical reamer head into the patient's body, and a path of sufficient width to accommodate a reamer head of the desired diameter must be cleared through the patient's body to the acetabulum. Thus, the amount of soft tissue trauma is directly proportional to the size of the reaming instrument inserted through such tissue.

It has recently become a goal for orthopedic surgeons to perform traditional surgeries in a less invasive manner. Such less invasive surgeries are called minimally invasive surgical (“MIS”) procedures. MIS procedures are performed in a manner that requires less cutting of a patient's soft tissues in order to properly install an endoprosthetic implant.

Given the above related correlation between the size of the head of an acetabular reamer and the amount of trauma to soft tissue, and the goal of pursuing MIS procedures in hip arthroplasty, it has become desirable to provide an acetabular reamer that can prepare a patient's acetabular cavity while at the same time requiring less trauma to the patient's surrounding soft tissues in order to accommodate the instrument.

### SUMMARY OF THE INVENTION

The present invention relates generally to an orthopaedic reaming instrument, more specifically the present invention relates to a low profile acetabular reamer for use in traditional and in minimally invasive surgical (“MIS”) procedures. The acetabular reamer of the present invention includes a convex arcuated cutting surface having a plurality of cutting teeth disposed thereon, wherein the cutting surface comprises a non-hemispherical shape. Although, the present reamer's cutting surface is not a complete hemisphere, when spun, it provides a hemispherical cutting profile.

An advantage of the present invention is that the width of the low profile reamer is less than the diameter of a traditional reamer of similar cutting size. Therefore, the present reamer is capable of fitting through a smaller incision than a fully hemispherical reamer of equivalent cutting radius.

These and other advantages of the present invention will be apparent to those skilled in the art upon an examination of the present specifications, drawings, and claims.

### BRIEF DESCRIPTION OF THE DRAWINGS

FIG. 1 is a perspective view of a prior art acetabular reamer.

FIG. 2 is a perspective view of a representative acetabular reamer according to the present invention.

FIG. 3 is a top view of a representative acetabular reamer according to the present invention.

FIG. 4 is a bottom view of an acetabular reamer according to one embodiment of the present invention.

FIG. 5 is a right/left side elevational view of a representative acetabular reamer according to one embodiment of the present invention.

FIG. 6 is a front/rear side elevational view of an acetabular reamer according to one embodiment of the present invention.

FIG. 7 is a perspective view of a cutting tooth.

### DETAILED DESCRIPTION OF THE INVENTION

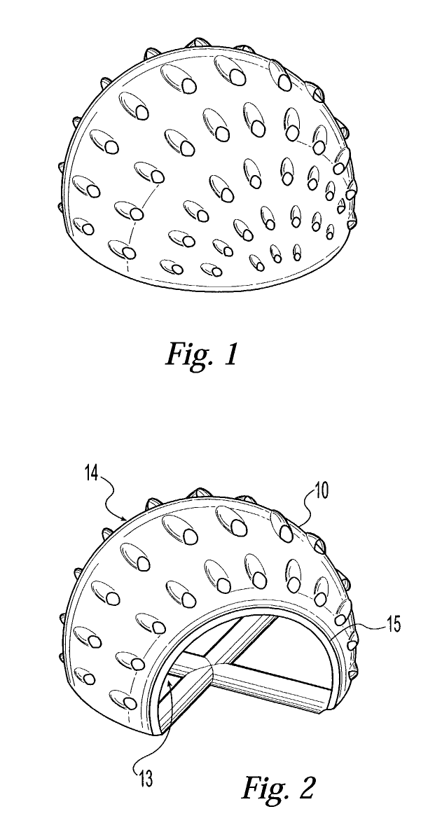

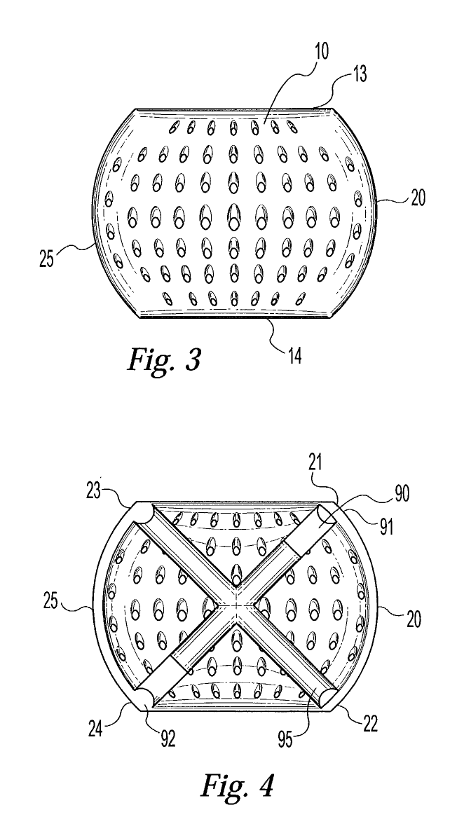

FIG. 1 shows a perspective view of a hemispherical acetabular reamer, as is known to those of skill in the art. Such reaming devices are typically removably attached to a powered surgical drill (not shown). The drill spins the reamer as a surgeon places the reamer against the acetabulum of a hip arthroplasty patient. The reamer creates a hemispherical cutting profile that the surgeon uses to create a substantially smooth arcuated acetabular cup area suitable for the receipt of an artificial acetabular shell to be used in the hip arthroplasty.

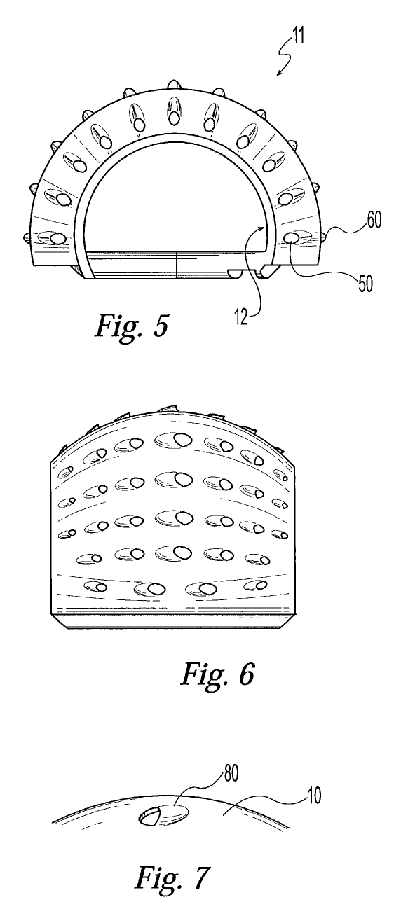

FIG. 2 shows a perspective view of a low profile acetabular reamer according to one embodiment of the present invention. The reamer of FIG. 2 comprises cutting surface The reamer may be constructed of any biocompatible and cleanable metal that is not prone to oxidation, such as titanium, or cobalt chrome. Preferably, however, the reamer is constructed of stainless steel. Cutting surface 10 comprises an arcuated convex surface generally in the shape of the central portion of a hemispherical shell, wherein the central portion has a first open side 13 and a second open side 14. As shown in FIG. 5, each side 13 and 14 are preferably open and have an arcuated top portion 15 corresponding to one edge of cutting surface 10. Cutting surface 10 further comprises a pair of opposing curved ends 20 and 25, shown in FIG. 4. Curved ends 20 and 25, preferably have an equal arc length and are disposed between the first and second sides of curved surface 10. Curved ends 20 and 25 comprise a radius of curvature preferably equal to that of cutting surface 10.

Referring again to FIG. 2, cutting surface 10 further comprises outside 11 and inside 12. A plurality of bores 50 is distributed in a desired pattern along cutting surface 10 such that each bore 50 is in communication with outside 11 and inside 12 of cutting surface 10. Cutting surface 10 still further comprises a plurality of cutting teeth 60 disposed on outside 11 of cutting surface 10, and adapted to cut human bone. Each cutting tooth 60 is disposed adjacent to and in communication with bore 50, such that there is a one to one ratio between cutting teeth 60 and bores 50.

Each cutting tooth 60 disposed on outside 11 of cutting surface 10 comprises a partially stamped portion of the material comprising cutting surface 10, such that a slight indention exists adjacent to and in communication with each bore 50 on inside 12 of cutting surface 10, while a slight raised cutting edge 80, as shown in FIGS. 6 and 7, exists adjacent to and in communication with each bore 50 on outside 11 of cutting surface 10. Cutting edges 80 are adapted and directed such that they are suitable to cut bone.

Referring now to FIG. 4, cutting surface 10 preferably further comprises reinforcement means. Such means preferably includes cross members 90 and 95. Cross members 90 and 95 are preferably constructed of the same material as cutting surface 10. Cross members 90 and 95 comprise a generally cylindrical shape. Cross member 90 further comprises opposing ends 91 and 92; cross member 95 further comprises opposing ends 96 and 97. As shown in FIG. 4, opposing curved edge 20 further comprises ends 21 and 22, and opposing curved edge 25 further comprises ends 23 and 24.

Each cross member 90 and 95 is statically connected to a diagonally opposing end of curved edge 20 and 25, such that, for example, end 91 of cross member 90 is connected to end 21 of curved edge 20 while end 92 of cross member 90 is connected to end 27 of curved edge 25. Similarly, end 96 of cross member 95 is connected to end 22 of curved edge 20, while end 97 of cross member 95 is connected to end 96 of curved edge 25.

Referring still to FIG. 4, at the midpoint of cross member 90 is notch 93. At the midpoint of cross member 95 is notch 98. Cross members 90 and 95 intersect at notches 93 and 98, which notches are adapted to interlock together. Cross members 90 and 95 are welded where notches 93 and 98 interlock.

The acetabular reamer of the present invention is generally releasably attached to a rotating means such as an elongate member attached to a surgical drill. Although the present acetabular reamer does not comprise a completely hemispherical shape, as it spins it is nevertheless capable of producing a hemispherical cutting profile.

The reamer is inserted into a patient's body and used to prepare the acetabulum of the patient to accept an artificial acetabular cup. Surgeons are able to advantageously insert the reamer of the present invention into smaller incisions than would be possible with a full hemispherical reamer, because the width of the reamer between is generally smaller than the diameter of a hemispherical reamer having a similar cutting profile.

We claim:

1. An acetabular reaming apparatus, comprising: a convex cutting surface, the cutting surface comprising a generally hemispherical shape having truncated opposing sides; a plurality of cutting teeth disposed on the cutting surface; and means for releasably attaching the apparatus device to means for spinning the apparatus.

2. The apparatus of claim 1, wherein the apparatus comprises stainless steel.

3. The apparatus of claim 1 or 2, wherein the cutting surface further comprises a non-semicircular cross-sectional area in at least one plane, said at least one plane being perpendicular to a plane tangent to the apex of the convex cutting surface.

4. An acetabular reaming apparatus, comprising: a pair of generally flat open opposing sides; a pair of curved opposing edges, wherein the curved edges are farther apart than the flat sides; a convex cutting surface disposed between the pair of flat sides and the pair of curved edges; and a plurality of cutting teeth disposed on the cutting surface.
